(19) United States
(12) Patent Application Publication
(10) Pub. No.: US 2008/0268547 A1
(43) Pub. Date: Oct. 30, 2008

(54) Systems and Methods for Indicating Oxidation of Consumer Products

(75) Inventors: Jason Avent, Austin, TX (US); Neel Naik, Sugar Land, TX (US); Tom J. Mabry, Austin, TX (US); Michael J. Passineau, Irondale, AL (US)

Correspondence Address: FISH & RICHARDSON PC, P.O. BOX 1022, MINNEAPOLIS, MN, 55440-1022, US

(73) Assignee: BOARD OF REGENTS, THE UNIVERSITY OF TEXAS SYSTEM, Austin, TX (US)

(21) Appl. No.: 11/547,763

(22) PCT Filed: Mar. 24, 2005
(86) PCT No.: PCT/US2005/009695

§ 371 Date: Jul. 1, 2008

Related U.S. Application Data
(60) Provisional application No. 60/561,043, filed on Apr. 9, 2004.

(51) Int. Cl. G01N 21/75; G01N 33/48; A61K 31/19; A61K 31/05; A61K 31/551; A61K 31/65; G01F 1/64

(52) U.S. Cl. 436/164; 422/68.1; 205/782; 426/87; 514/570; 514/152; 514/734; 514/221

(57) ABSTRACT

The present invention relates to systems and methods using colorimetric indicators of oxidation in consumer products. In particular, betalains are used as oxidation indicators in food, pharmaceutical, and other products.

### FIELD OF THE INVENTION

The present invention relates to systems and methods for using colorimetric indicators of oxidation in consumer products.

### BACKGROUND OF THE INVENTION

Expiration dates for consumable products have been in use for decades. The current system of expiration dates assigns one relatively conservative estimate of product life prior to opening. These products may encounter very different environmental insults after distribution. Conservative expiration dates encourage waste and, conversely, numerous products often degrade while still within the expiration date. The current system exposes manufacturers and retailers to liability and the printed expiration information is frequently not utilized, seen or understood by consumers.

Also currently needed is a system that can provide quality assurance to the consumer even after a product has been opened and over the course of consumption. Currently, pharmacists use a one year “rule of thumb expiration” on dispensed prescription medicines even while their storage bottles of pills have been repeatedly opened and exposed to multiple oxygen and humidity insults. More often than not, the expiration date is not adequate for alerting consumers to the quality of their products, and the current system does not provide adequate assurance to the consumer after purchase.

Medications, cosmetics, and sunscreens use expiration dates to inform the consumer as to when the unopened product is no longer suitable for use. Many of these products remain acceptably safe even after the expiration date, and these otherwise useful products are prematurely discarded. For example, if a degradable sunscreen product loses its protective qualities, there is typically no warning to the user, thereby resulting in excessive sun exposure, freckling, and an increased risk of skin cancer.

Expired drugs and herbal medications can pose serious health and environmental risks due to the potential loss of active ingredient and the creation of degraded drug derivatives that have unknown or unwanted side effects. For example, degraded tetracycline is known to cause kidney damage. FDA testing of pharmaceuticals typically insures the safety of the drug molecule and its metabolites, not its degradation products. Additionally, other components of pharmaceutical formulations may be susceptible to oxidative degradation, rendering the formulation ineffective as designed. For example, oxidative damage to the matrix of controlled-release formulations can reduce the ability of these formulations to work as intended. Accordingly, there is a need to provide the ability to substantially eliminate consumer exposure to oxidized pharmaceuticals.

Colorimetric indicators have found applications in many aspects of science and consumer goods. Examples include pH papers, Elisa assay tests like the urine pregnancy test, and calorimetric reagents used by law enforcement to identify narcotics. These tests require a control reaction to assure that reagents are still active and they require destruction of the sample to be tested. Accordingly, there is a need for a calorimetric indicator that involves passive visual observation of the product either directly or through a transparent portion of the packaging. In this way, no control reaction would be necessary, and the product would not be compromised or destroyed in testing.

Betalains are natural colorants that are believed to have been utilized by plants as attractants and/or ripeness indicators for millions of years. Betalains color flowers and fruits to attract pollinators and seed dispersers. Once the seed matures and the fruit ripens the betalain is produced allowing vertebrates to spot the nutritious, attractive fruit. Betalain pigmentation of flowers and fruits provides an example of a coevolution mutualism benefiting both the plant and the animal. As long as the animal does not destroy all seeds and the plant provides non-toxic nutritious fruits, the mutualism is maintained. Betalains are colorants first used by plants then adopted by people to make products look more pleasant, consistent, and appetizing. Betalain pigments are one of the natural world's indicators of “ready to eat.”

Betalain is well suited to be an additive. It is flavorless, odorless, naturally-occurring, and GRAS (generally recognized as safe). Betalains are charged antioxidants that have substantially proven their effectiveness as antioxidants. Inside the digestive tract of rats most betalain is degraded by free radicals, providing protection to the cells of the stomach, small intestine, and colon from free radical damage. A percentage of betalains are absorbed into the bloodstream, circulated through the body, and filtered out by the kidneys. Accordingly, these circulating molecules have the capacity to neutralize free radicals even outside the digestive tract. Betalain is a beneficial part of the diet that has been found safe and beneficial in many scientific studies. Its natural derivation and long history of safe use have exempted it from regulation even in systems as rigorous as the German food and drug industry where purity and safety are tightly controlled.

Characterization and use of betalains in various consumables are reported in, for example, Buettner G R. Arch Biochem Biophys. 1993, 300(2), 535; Butera D, et al. J Agric Food Chem. 2002; 50(23), 6895; Cai Y et al., J Agric Food Chem. 2003, 51(8), 2288; Delgado-Vargas F, et al. Crit Rev Food Sci Nutr. 2000, 40(3), 173; Kanner J, et al. J Agric Food Chem. 2001, 49(11), 5178; Mabry T J. J Nat Prod. 2001, 64(12), 1596. Waterman K C, et al. Pharm Dev Technol. 2002, 7(1), 1. Methods of isolating, purifying and using betalain for use as colorants, sunscreens and nutraceuticals are reported in U.S. Pat. Nos. 4,027,042; 4,115,595; 4,132,793; 4,238,518; 4,409,254; 4,442,104; 5,089,410; 6,379,729; and 6,783,754; as well as WO 97/46622 and WO 98/26972. Indicators used for detecting freshness of food items are reported in U.S. Pat. No. 6,325,974.

Accordingly, there is a present need to provide an indicator of preservation status (e.g. presence or absence of oxidative changes), particularly for consumable products, which is non-toxic, reliable, and easily implemented. The calorimetric indicator systems and processes described herein are directed toward this end.

### SUMMARY OF THE INVENTION

The present invention provides systems for determining the presence or absence of oxidative changes in an article, comprising: a) an article which is subject to degradation or spoilage in the presence of air or oxidative conditions; b) a colorimetric indicator comprising a colored compound which changes color upon oxidation; wherein the indicator is exposed to substantially the same environmental conditions as the article; and c) a reference standard, whereby comparison of the color of the indicator with the reference standard determines the presence or absence of oxidative changes in the article. In some embodiments, the article and the indicator are combined together. In some embodiments, the article and the indicator are enclosed together within a packaging. In some embodiments, the reference standard is disposed on the packaging or referred to on the labeling of the packaging.

In some embodiments, the indicator is derived from a natural source. In some embodiments, the indicator comprises a betalain such as a betacyanin or derivative thereof or a betaxanthin or derivative thereof. In some embodiments, the indicator is derived from fungi of the family Amanita or plants of the order Caryophyllales. In some embodiments, the indicator is derived from a plant of the family Chenopodiaceae, Cactaceae, Amaranthaceae, Nyctaginaceae, Aizoaceae, Basellaceae, Didieraceae, Phytolaccaceae, or Portulaceae. In some embodiments, the indicator is derived from amaranth, Malabar spinach fruit, red beets, prickly pear, Phytolacca americana, Capitatum berries (Chenopodium capitatum), or hottentot fig (Carpobrotus edulis). In some embodiments, the indicator is in liquid form. In some embodiments, the indicator is in solid form. In some embodiments, the indicator is disposed on a solid substrate.

In some embodiments, the systems of the invention further comprise an electrolyte. In some embodiments, the electrolyte is in contact with both the indicator and the article.

In some embodiments, the systems of the invention further comprise a primary reductant. In some embodiments, the primary reductant is a stronger reductant than the colored compound or a substantially equal reductant to the colored compound. In some embodiments, the primary reductant is ascorbic acid, catenin, tocopherol, tocotrienol, or mixture thereof.

In some embodiments, the article is a pharmaceutical, a natural product, a food, or a skin treatment. In some embodiments, the pharmaceutical comprises ibuprofen, tetracycline, a statin, amphoteracin B, hydroquinone, tetrazepam, or mixture thereof.

In some embodiments, the reference standard comprises a graphic indication, textual indication, or combination thereof of the presence or absence of oxidative changes in the article. In some embodiments, the graphic indication comprises one or more colors corresponding to substantially unoxidized, substantially oxidized, or partially oxidized indicator. In some embodiments, the textual indication comprises one or more words or phrases describing the color of substantially unoxidized, substantially oxidized, or partially oxidized indicator. In some embodiments, the reference standard comprises spectrophotometric information.

The present invention further provides systems for determining the presence or absence of oxidative changes in a pharmaceutical, comprising: a) a pharmaceutical composition containing a component which is subject to degradation in the presence of air or oxidative conditions; b) a calorimetric indicator comprising a betalain, wherein the indicator is exposed to substantially the same environmental conditions as the pharmaceutical composition; and c) optionally a reference standard, whereby comparison of the color of the indicator with the reference standard determines the presence or absence of oxidative changes in the pharmaceutical. In some embodiments, the component is an active pharmaceutical ingredient (API).

The present invention further provides liquid pharmaceutical compositions comprising: a) an active pharmaceutical ingredient (API); b) a calorimetric indicator comprising a betalain; and c) a liquid excipient. In some embodiments, the liquid pharmaceutical composition is in oral dosage form. In some embodiments, the oral dosage form is a capsule containing the liquid pharmaceutical. In some embodiments, the capsule is transparent. In some embodiments, the liquid pharmaceutical composition further comprises an electrolyte. The liquid pharmaceutical composition of further comprises a primary reductant. In some embodiments, the API is ibuprofen.

The present invention further provides solid pharmaceutical compositions comprising: a) an active pharmaceutical ingredient (API); b) a calorimetric indicator comprising a betalain; and c) a solid excipient. In some embodiments, the solid pharmaceutical composition is in oral dosage form. In some embodiments, the solid pharmaceutical composition is contained within a capsule. In some embodiments, the capsule is transparent. In some embodiments, the solid pharmaceutical composition further comprises an electrolyte. In some embodiments, the solid pharmaceutical composition further comprises a primary reductant.

The present invention further provides a packaged food comprising: a) food enclosed within a packaging; and b) a calorimetric indicator which is enclosed within the packaging together with the food, wherein the indicator is separate from the food, and wherein the indicator comprises a betalain. In some embodiments, the packaged food has an intended shelf life without refrigeration of greater than about 1 year. In some embodiments, the food is hermetically sealed within the packaging. In some embodiments, the indicator is in solid form. In some embodiments, the indicator is disposed on a solid substrate. In some embodiments, the packaged food further comprises a reference standard whereby comparison of the color of the indicator with the reference standard is determinative of the presence or absence of oxidative changes in the food. In some embodiments, the packaged food is a meal-ready to-eat (MRE).

The present invention further provides methods of determining the presence or absence of oxidative changes in an article, comprising comparing the oxidative changes of a calorimetric indicator comprising a betalain with a reference standard, wherein the indicator is exposed to substantially the same environmental conditions as the article; whereby the comparison indicates the presence or absence of oxidative changes in of the article. In some embodiments, the comparing is conducted by visually inspecting the color of the indicator and visually inspecting one or more colors graphically or textually indicated on the reference standard. In some embodiments, the comparing is conducted using spectrophotometric methods. In some embodiments, the comparing is conducted using electrochemical methods. In some embodiments, the comparing is automated.

The present invention further provides methods of purifying betalain comprising filtering a plant extract comprising betalain, wherein the filtering comprises cross-flow membrane filtration technology. In some embodiments, the plant extract undergoes microfiltration, ultrafiltration, nanofiltration, reverse osmosis, demineralization, or combinations thereof. In some embodiments, the method comprises subjecting the extract or partially purified form thereof to fermentation. In some embodiments, the method further comprises quantifying the betalain using spectroscopy.

### BRIEF DESCRIPTION OF THE DRAWINGS

FIG. 1 is a graphical representation of the degradation of a pharmaceutical product over time.

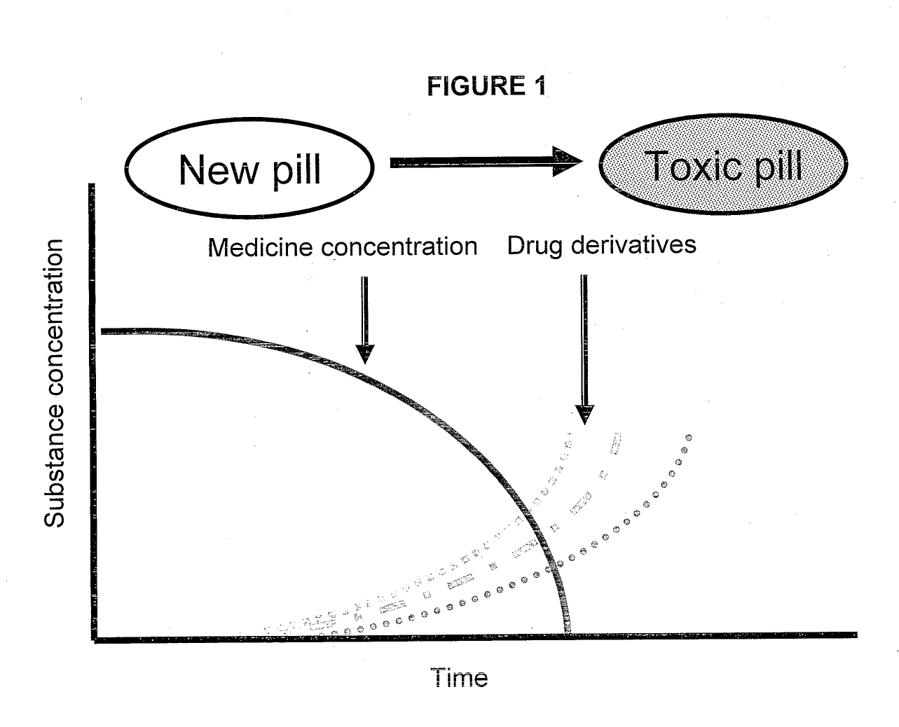

FIG. 2 is a graphical representation of the degradation of a pharmaceutical product containing betalain as an indicator of oxidation over time.

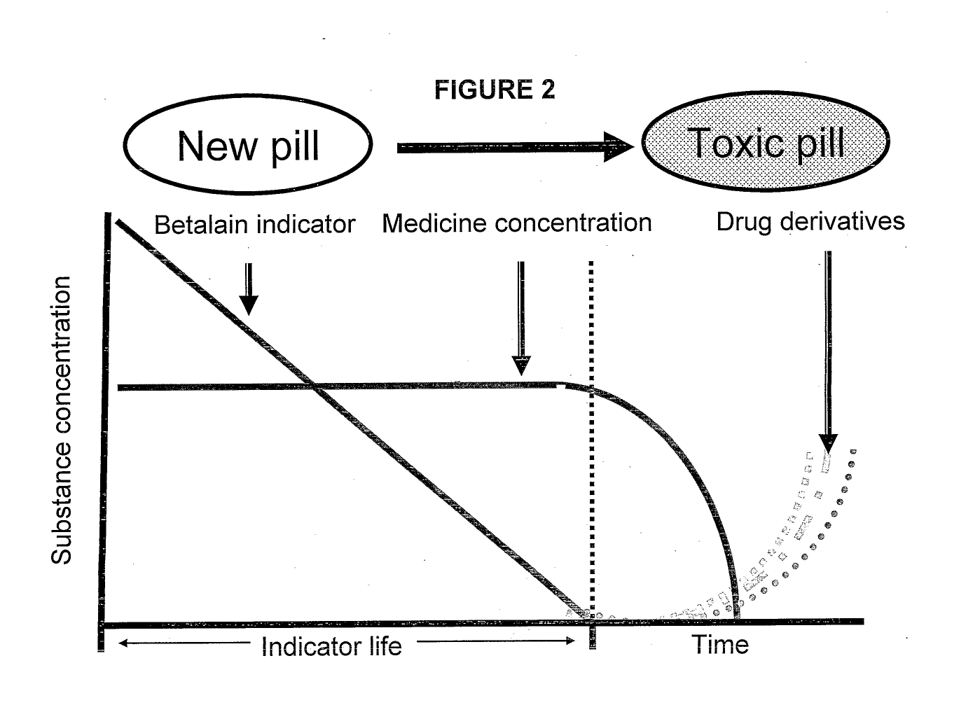

FIG. 3 is a graphical representation of the degradation of a pharmaceutical product containing a primary reductant and betalain as an indicator of oxidation over time.

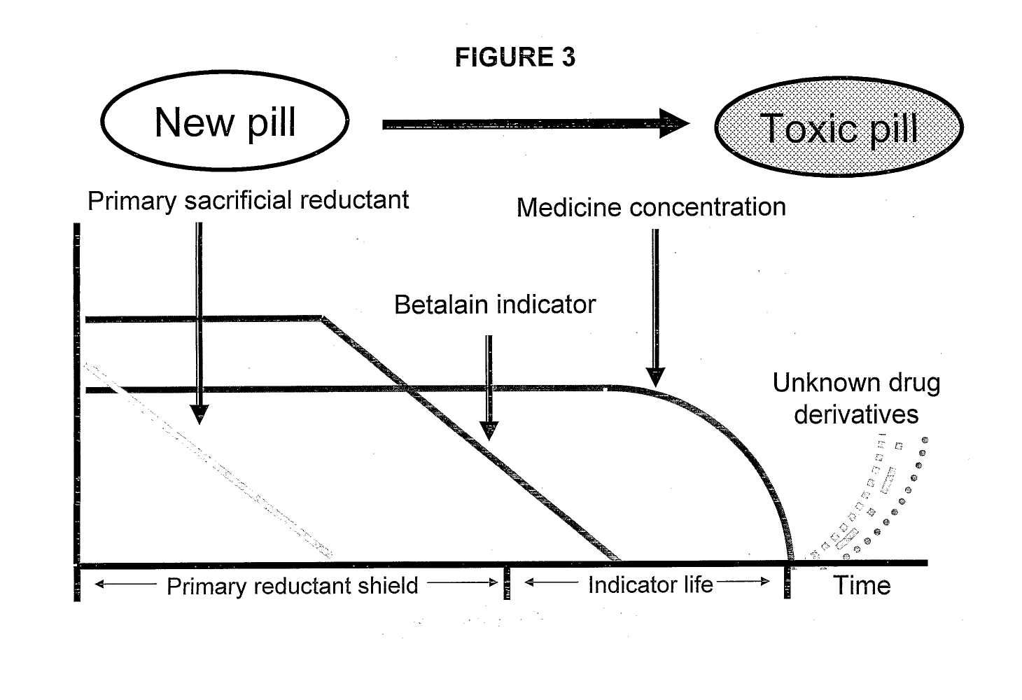

FIG. 4 is a graphical representation of a reference standard in the form of a color bar.

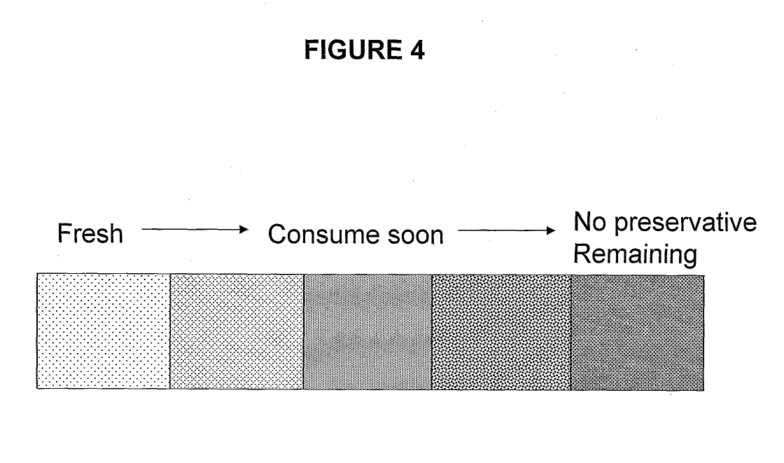

### DETAILED DESCRIPTION

The present invention provides, inter alia, methods and systems for indicating the presence or absence of oxidation in an article which is sensitive to degradation or spoilage upon exposure to air or oxidative conditions. The systems and methods of the invention employ a calorimetric indicator which contains a colored compound capable of changing color when oxidized. The color change can be compared with a reference standard which can indicate exposure of the article to oxidative conditions that would lead to degradation and/or spoilage.

The calorimetric indicator can contain one or more colored compounds that visibly change color when the compound becomes oxidized, for example by exposure to dioxygen or other oxygen-containing oxidizing species such as ozone, peroxides, hydroxyl radical, superoxide, and the like. The indicator can detect the presence or absence of oxidative changes in an article. The phrase “oxidative changes” is meant to include both oxidative degradation by chemical reaction of the article or component thereof with an oxidizing species as well as oxidative exposure, where the article is subjected to oxidative conditions but not necessarily degraded.

Suitable colored compounds include both organics and inorganics. In some embodiments, the colored compound is an organic compound which is derived from a natural source such as fungi or plants. For example, the indicator can include betalain, a natural colorant found in fungi of the family Amanita as well as many plants of the order Caryophyllales. Betalain producing plants are found in the families Chenopodiaceae (red beet), Cactaceae (cactus, e.g., prickly pear), Amaranthaceae (amaranth), Nyctaginaceae, Aizoaceae, Basellaceae (Malabar spinach), Didieraceae, Phytolaccaceae, and Portulaceae. Betalains are found in all parts of the plant including leaves, stems, flowers, fruits, seeds, and roots. In some embodiments of the invention, the betalain is derived from amaranth, Malabar spinach fruit, red beets, prickly pear, Phytolacca americana, Capitatum berries, or hottentot fig. In further embodiments, the betalain is derived from Malabar spinach fruit.

Betalain refers to a family of nitrogen-containing organic pigments which include the red or red-violet betacyanins and the yellow or orange betaxanthins. Accordingly, the term “betalain” or “betalains,” as used herein, is employed in accordance with the art and refers to one or more betalamic acid derivatives including any one or more betacyanins, betaxanthins, acylated or other derivatives thereof or combinations thereof. Example betacyanins include betanin, isobetanin, neobetanin (14,15-dehydrobetanin), prebetanin, and the like. Example betaxanthins include vulgaxanthin-I, vulgaxanthin-II, indicaxanthin, and the like. Further, example betalains can be prepared by the condensation of amines (e.g., amino acids) with betalamic acid to form corresponding colored imine or immonium analogs of betacyanins and betaxanthins. Some example betalains are described in the art such as, for example, in Trezzini et al., Phytochemistry, 1991, 30(6), 1897 and Schliemann et al., Phytochemistry, 2001, 58, 159, each of which is incorporated herein by reference in its entirety.

Betalain is oxygen sensitive and oxidized in air. The oxidation of betalain typically leads to loss of its vivid color, rendering dull brown or substantially colorless compounds. Accordingly, this color change can be detected visually or by spectrophotometric methods (e.g., absorption, emission, fluorescence). Because the color change corresponds to oxidation of the molecule, the change can also be detected by electrochemical methods. In some embodiments, the betalain has a red or red-violet color which changes to brown upon oxidation. In systems exposed to heavy oxidation, even the brown color can be bleached to substantially no color.

Betalains can be derived from natural sources such as plant extracts which can be used raw or purified (either substantially or partially). Betalains can also be derived from recombinant DNA sources and can also be synthesized by, for example, condensation of betalamic acid (or derivatives and isomers thereof) with amines.

Betalains exert a protective effect on other substances with lesser capacity for oxidation such as by radical absorption. Such substances have a standard electrode potential less than (more negative) the betalain pigment, and therefore are candidates for direct protection by the pigment. Substances with an electrode potential greater than the pigment can also be utilized according to this invention, but benefit from use of a calibrated indicator system separate from the product. In this context, the indicator can preserve weaker antioxidants (e.g., those with lower electrode potentials) until the indicator is almost completely depleted by oxidation. This effect is referred to in the art as a sacrificial reductant effect, particularly with respect to the art of pharmaceuticals. Sacrificial reductants are often called preservatives and also referred to herein as “primary reductants.” Because betalain changes color from a bright hue to a dull brown or colorless state upon oxidation, it can serve as a calorimetric indicator of oxidation status as well as a preservative. Betalains have the capacity to act as antioxidants by preferentially taking on oxidative damage, for example, by terminating the chain reaction of radical damage and thereby slowing the rate of oxidation.

Determining the oxidation of a non-colored sacrificial reductant in, for example, a pharmaceutical preparation can be difficult and expensive without an indicator. Betalain can act as a sacrificial reductant as well as an indicator. After absorbing the damaging effects of free radicals, betalain pigments change from an initial bright color (purple, red-violet, red, orange, or yellow) to brown or no color. This color change can be used as a calorimetric indicator which reveals the exposure of the product to oxidative damage and/or depletion of any other preservative in the product.

Betalain can be incorporated within the product and typically begins as a brightly colored compound. When exposed to oxidizing conditions such as free radical damage, the betalain shifts to a brownish color or no color. If the product can be observed through the packaging then the evaluation can be done non-destructively, e.g., prior to opening. False negatives are made difficult because of the contrast in color of the two states. Additionally, the color change matches the intuitive preconceptions of good (brightly colored) versus bad (brown).

The evaluation of color change in a betalain-containing indictor can be obvious to a consumer who fails to look at the packaging, but notices that the color of the product has changed since its first use. This observation can cause the consumer to consult a product label which can exhibit a reference standard such as a color indicator bar that allows comparison with product color. The color bar can optionally be marked with pictograms of good or bad (e.g., Mr. Yuck) for illiterate or users. Even though assurance of non-oxidation is diminished when betalain becomes brown, the product is can still be considered acceptable, theoretically, until the last betalain molecule is consumed. Accordingly, the calorimetric indicator function of betalain is a vast improvement over typically hard-to-read expiration dates that require consumer vigilance to be effective.

Compounds other than betalains suitable as indicators in the context of the invention include, for example, other naturally-derived pigments such as anthocyanins, carotenoids (e.g., lycopene) which have been observed to fade with oxidation.

The indicator of the systems and methods of the invention serves to detect oxidation of the article or oxidation in the environment of the article. Thus, the indicator typically is subjected to the same or substantially the same environmental conditions as the article, so that the color of the indicator is a true measure of the article's degradation or exposure. In some embodiments, the indicator and the article are combined together, meaning they are held together, although not necessarily in contact with each other, such as by a linkage or enclosure. In some embodiments, the indicator and the article are combined together within a packaging such as a container, bottle, bag, pouch, can, capsule and the like. In this way, both the indicator and article are subject to the same or similar environmental conditions experienced within the enclosure or packaging.

The article can be any item that can suffer degradation or spoilage upon exposure to air or other oxidative conditions. Example articles include articles containing pharmaceuticals (e.g., in dosage forms), articles containing natural products or synthetic compounds, articles which are foods or foodstuffs, articles which are biological samples or reagents, or articles which are skin treatments.

Many components of pharmaceutical formulations, including the active pharmaceutical ingredient (API), are known to decompose upon exposure to an oxidative environment (e.g., air). See, e.g., Hovorka, et al. Journal of Pharmaceutical Sciences, 2001, 90(3), 253; Waterman et al., Pharmaceutical Development and Technology, 2002, 7(1), 1; and Karki, et al., J. Pharm. Sci., 2000, 89(12) 1518; each of which is incorporated herein by reference in its entirety. Accordingly, such pharmaceuticals can have a limited shelf-life, even when optimally formulated and packaged under the most stringent of conditions. Example active pharmaceutical ingredients which can oxidatively degrade include ibuprofen, tetracycline, statins, amphoteracin B, hydroquinone, tetrazepam, and the like. Other suitable APIs include antibiotics, antitoxins, vaccines, chemotherapeutic agents, and the like. Still other suitable APIs include protein or nucleic acid drugs such as antibodies or antisense and interference RNA compounds. A drug's susceptibility to oxidative degradation can be measured by routine methods in the art including, for example, free radical exposure strategies.

Pharmaceutical articles, according to the invention, can be provided in dosage form. Any type of dosage form is suitable. Example dosage forms include oral dosage forms such as tablets, capsules, troches, lozenges and oral liquids, suspensions or solutions. In some embodiments, the dosage form is a capsule including, for example, a liquid gel capsule.

As with pharmaceuticals, natural products such as a nutritional supplements and natural remedies are often subject to oxidative degradation. Example natural products include herbal extracts, tinctures, decoctions, teas, syrups, tonics, toddy, powdered extract, fresh plant, dried plants, fungus or bacterial products. A further natural product is St. John's Wort or a component thereof such as hypericin or hyperforin. A further natural product is S-adenosyl methionine. These are just a few of the medicinal and other such concentrated health promoting formulations which usually contain an active ingredient extracted from the plant. Other natural products include nutraceuticals which are purified, quantified, balanced and engineered versions of the herbal medicine. Accordingly, articles of the present invention include dosage forms containing an oxidatively degradable natural product.

It is well known that foods, beverages, and foodstuffs (collectively “food”) can spoil upon exposure to air. Any food is suitable for use with the indicator systems of the invention. In some embodiments, the food or beverage is intended for long term storage, for months or even years. Further addition of a primary reductant such as ascorbic acid to the food can delay the onset of indicator color change. Food articles of the invention can have product lifespans tailored for up to about 5 years, up to about 4 years, up to about 3 years, up to about 2 years, or up to about 1 year as desired by the manufacturer and is practical for the market. Example foods include jarred foods, freeze-dried foods, vacuum-packed foods, nitrogen-packed foods, frozen foods, and the like. In some embodiments, the food is packaged in the form of a meal-ready-to-eat (MRE). In further embodiments, the food is packaged for emergency or camping use. In yet further embodiments, the food can be in the form of a frozen dinners. Here, there is an additional visual indication of a freeze-thaw cycle as the indicator could “bleed color” into another compartment upon thawing. Additionally, the food items of a single frozen dinner can be calibrated to have similar oxidation rates and capacity so that one item in the dinner could act as a calibrated indicator system for all of the food items contained inside.

Topical skin treatments, including cosmetics, sun protectants (e.g., sunscreens and sunblocks), and skin sanitizers (e.g., hand sanitizers) often have active ingredients that can degrade upon prolonged exposure to air and could benefit from use of an indicator of oxidation status. For example, a betalain-pigmented (e.g., orange) sunscreen with optionally added titanium dioxide could act as a skin tone cosmetic and could reveal the loss of sun protective efficacy by turning brown. In other embodiments, betalain can allow a visual check of complete skin coverage of the sunscreen or other skin treatment. Any color due to betalain in the skin product can be washed away upon immersion in water as betalain is water-soluble; leaving absorbed to the skin the sunscreen and/or skin conditioners. Additionally, the color of betalain pigments will allow evaluation of complete skin coverage.

Articles can also include reagents and products used in life sciences, medicine, and related fields. Example reagents and products include but are not limited to nucleic acids, proteins, reagents, enzymes, antibodies, cell lines and buffers. These stocks and components can be colored with indicator and can optionally contain a preservative (e.g, primary reductant). When indicator color changes, additional preservative (e.g., primary reductant) and indicator can be added to valuable stocks to extend their life. In addition to the indication of oxidation, the colored indicator can be used as a marker provide confirmation that a solution has been added to a mixture.

The indicator can be provided in liquid form where, for example, the colored compound is dissolved, dispersed or suspended in a liquid media. The term “liquid” as used herein is meant to describe media having fluidic or malleable properties including, not only water and solvents, but also oils, emulsions, waxes, pastes, creams, and the like. Betalain can be made to dissolve into oils through derivitization, emulsification, or combination with charged compounds. In some embodiments, the liquid media is a pharmaceutically acceptable carrier liquid such as water, primary alcohols (e.g., ethanol), polyols (e.g., glycerol, propylene glycol and liquid polyethylene glycol), suitable mixtures thereof, and vegetable oils, or combinations thereof. Example liquid indicators include plant extracts containing betalain. The extracts can be raw juice, purified or partially purified betalain pigments. Additionally, the liquid component of an extract can be removed and replaced with another liquid.

The indicator can also be provided in solid form. For example, the colored compound, such as betalain, can be provided as a purified, or partially purified solid. The solid can be amorphous, crystalline or combination thereof. The solid can contain additional substances, such as co-extracted compounds, minerals, or salts. Solid betalain can be obtained, for example, by lyophillization of an extract containing betalain. In some embodiments, solid betalain can be obtained by spray drying, optionally together with one or more carriers such as soluble starch, gum acacia, maltodetrine, and the like as described, for example in Koul, V. K. et al., Indian Journal of Chemical Technology, 2002, 9(5), 442-445, which is incorporated herein by reference in its entirety.

Solid indicator further includes solid substrates onto which the colored compound is disposed. For example, betalain can be disposed on a solid substrate where the betalain is, for example, chemically attached to (e.g., by covalent bonds), adhered to, or trapped within a solid. Various solid substrates suitable for supporting an indictor are known to those of ordinary skill in the art. Examples of such substrates include paper (e.g., celluloses including untreated cellulose), polyamides, cellulose acetate, hydroxypropyl methyl cellulose, gellan gum, agar, gels, foams, glass (e.g., glass fibers) and resins such as ion-exchange resins. Waxes and related substances can also be infused with an indicator. The solid substrates of the present invention typically are planar and optionally flexible with a thickness of less than about 1 mm. Indicator disposed on solid support can be used on the interior surface of packaging, capsules, or other product container. In other embodiments, the supported indicator can be placed on the exterior of a capsule or some other container for the article. In further embodiments, the article packaging can serve as the solid support, for example, where the indicator is incorporated within and throughout the packaging (e.g., a capsule) or other container for the article. In some embodiments, betalain which is covalently bound to a solid substrate can act as an indicator for an adjacent oil-based article.

Methods of affixing indicator to solid substrate are known in the art. In particular, betalains can be attached to solid substrate directly through the betalain functional groups (e.g., hydroxyls, amines, etc.) or indirectly through a linker moiety (e.g., saccharides, oligosaccharides, tannins, tannic acids, phenols, polyphenols, etc.). In some embodiments, an amino (e.g., secondary amine) moiety of the indicator can be coupled to appropriately derivatized celluloses such as hydroxypropyl methylcellulose using routine methods. In addition, the betalamic acid, cyclodopa, glucose, or other moiety of the indicator can offer linkage sites for attachment to solid support via acylation or ether formation. For example, enzymatic or chemical processes can be used to react the pendant carboxyl and/or OH groups of the indicator with solid substrate, thereby immobilizing the pigment while still allowing it to retain its indicator properties.

In some embodiments, where the article is contained in a dry environment like the interior of a medicine bottle, a paper insert in the lid or other portion of the container could be affixed with indicator and optionally calibrated with any preservative (e.g., primary reductant) used in the article. In this situation, the indicator affixed to the paper would still be subject to the environmental insults that the product experiences, and thus can still function as an accurate indicator of remaining product life.

The indicator and the article can be in direct contact with each other, or can be separated from each other but subject to the same or similar environmental conditions. When the article and indicator are separated, a physical barrier is optional. For example, the article and indicator can be enclosed together within a container, but are immobilized such that they do not touch. In some embodiments, the article and indicator are separated by an oxygen permeable barrier such as a membrane. In this way, the indicator does not discolor or otherwise contaminate the article, yet both are capable of being exposed to the same oxidative damage. Example barriers include waxes, plastics and other polymers such as, for example, polyvinyl chloride and polyethylenes. The barrier thickness can be less than about 1 mm.

In a further embodiment, a portion or component of the article can contain indicator (optionally with added preservative) while other components in the same package lack indicator. In this way, the indicator-containing component can be used as a proxy for its indicator-lacking counterparts throughout the process of consumption. An example would be a bottle of pills where at least one pill contained indicator and the remainder did not. The indicator-lacking pills could be consumed preferentially over the indicator-containing pills until the bottle is used up or the indicator-containing pills indicate oxidative degradation.

According to some embodiments of the invention, the systems and methods for indicating oxidative status or exposure can further include an electrolyte which serves to facilitate communication of electrons from the article to the indicator (or vice versa). The electrolyte can be dissolved in the liquid component of either or both the article or indicator. Alternatively, the electrolyte can be mixed as a solid with solid indicator and/or solid article. Suitable electrolytes include any type of charged compound or salt such as, for example, alkali metal halides, alkaline earth metal halides, alkali metal nitrates, alkali metal nitrites, alkali metal sulfates, alkali metal sulfites, and the like. In some embodiments, where the article and the indicator are in contact and in liquid containing electrolyte, resistivity is kept low, thereby facilitating accurate indication of product quality throughout the article.

According to further embodiments of the invention, the systems and methods for indicating oxidation can further include a primary reductant. As used here, “primary reductant” is meant to refer to a reducing agent that is a stronger or substantially equal reductant compared with the colored compound of the indicator. While the colored compound by its nature is a reductant, and therefore a preservative, it is often desirable to include a primary reductant with the article that would protect the article upon exposure to air or oxidative conditions. When the primary reductant is a more powerful reductant than the colored compound, the primary reductant is used up preferentially and the indicator will indicate when the primary reductant has been consumed. When the primary reductant has about the same reducing strength as the colored compounds, both the primary reductant and colored compound will be used up at similar rates and the indicator will indicate consumption of the primary reductant through a more gradual change of color. Example primary reductants include, for example, tocotrienols, tocopherols, catenins, tea extracts, proanthocyanidins ascorbic acid, acetyl cysteine, adipic acid, ascorbyl palmitate, butylated hydroxyanisole (BHA), butylated hydroxytoluene (BHT), citric acid, cysteine, disodium editic acid (EDTA), dithiothreitol, fumaric acid, glutamic acid, malic acid, propyl gallate, sodium formaldehyde sulfoxylate, sodium bisulfite, sodium metabisulfite, sodium sulfite, sodium thiosulfate, tartaric acid, thioglycerol, thioglycolic acid, thiourea, toluene sulfonic acid or combinations thereof.

Primary reductants (e.g., primary sacrificial reductants or preservatives) are typically better (e.g., more active) free radical scavengers than the indicator. Accordingly, the primary reductant of greater potency can be layered above the indicator to allow extended lifetimes as desired by the manufacturer. Natural antioxidants such as vitamin C (ascorbic acid) and Vitamin E (tocopherols and tocotrienols) or other potent antioxidants can be used as primary reductants and will allow calibration of shelf life (while adding nutritional enrichment). These primary reductants can provide additional time between product manufacture and the beginning of indicator color change. Once the color does begin to change the consumer can be encouraged to use the product quickly; as the product can remain fully preserved until the color is completely converted.

Primary reductants can also be used to calibrate simultaneously degrading articles or components of an article (e.g., foods in an MRE) such that an indicator in one component can serve to indicate the oxidation status of the remainder components. Calibration can be carried out by combining indicator with all components of an article and then subjecting the indicator-containing components to oxidative conditions. Some components due to, for example, water content will degrade more quickly than others. Accordingly, an amount of primary reductant can be measured for each article component that would allow similar rates of degradation and the use of indicator in only one component to indicate the oxidation status of all. As an example of calibration, all food items in an MRE can be colored with betalain, and the moist items supplemented with primary reductant such as vitamin C and/or E. If the levels of primary reductant are sufficient, all of the foods will change their color substantially simultaneously upon exposure to air. Otherwise, more or less primary reductant is needed. Simultaneous color change indicates the items are calibrated. In a further embodiment, where large quantities of article are produced, one article can contain betalain and all others simply receive primary reductant. The betalain colored product serves to determine the oxidative status of the entire batch. This same procedure could be used to calibrate any part of a multi-component article, and an assurance of quality can be made instead of the estimation and guesswork that currently prevails in expiration dating. In some embodiments, the calibrated indicator can be set to any desired endpoint of oxidation in the article, allowing for some acceptable level of oxidation.

Numerous embodiments of the invention can be contemplated by combining the elements of indicator, article, optionally electrolyte, and optionally primary reductant. In some embodiments of the invention, indicator, article, and optionally primary reductant are combined together with an electrolyte. In this type of system, indicator coloration reveals a substantially unoxidized article when the rate of oxidation is not so rapid that it overcomes the rate of electron flow through electrolyte and diffusion of the primary reductant. In a second embodiment, indicator is bound to a solid substrate and is in contact with the article. In a third embodiment, the indicator and the article are separate from each other but are held together within a package such as a container, bottle, bag, pouch, can, capsule and the like. In this way, both the indicator and article are subject to very similar environmental conditions within the enclosure or packaging.

The systems and processes of the invention can further include a reference standard for comparison with the indicator to establish the presence or absence of oxidative changes in the article. The reference standard can be in any form and can be physically connected with the indicator, article, or article's packaging or merely referred to by the system or process. For example, the reference standard can be disposed on the article or on the article's packaging in the form of a label; or, an indication of where one would find the reference standard (e.g., in a reference book, on a web site, etc.) can be substituted for the actual reference standard on the package.

In some embodiments, the reference standard can include a graphic indication, textual indication, or combination thereof connecting the color of the indicator with the oxidation status or oxidative exposure of the article. A graphic indication can contain one or more colors, each corresponding to 1) substantially unoxidized indicator which would indicate that the article remained unoxidized, 2) substantially oxidized indicator which would indicate that the article suffered oxidative degradation or exposure to such conditions, 3) partially oxidized indicator which would indicate that the article will soon be oxidatively degraded, or 4) other visible change. In place of or in addition to colors, the reference standard can have one or more words, symbols or phrases describing the color of the indicator and/or conveying whether the indicator color corresponds to a preserved article or oxidatively degraded article.

In some embodiments, the reference standard contains spectrophotometric information such as data associated with absorption, emission, or fluorescence spectra (e.g., from near infrared to ultraviolet or a subrange thereof). For example, the absorption data of the indicator can be compared with a reference standard corresponding to an absorption spectrum of substantially oxidized, substantially unoxidized, or partially oxidized indicator. In further embodiments, absorption or emission at a preselected wavelength can be measured/monitored, where the preselected wavelength has varying signal strength depending on whether the indicator is oxidized or unoxidized or somewhere inbetween.

The present invention further provides methods of determining oxidative changes in an article by comparing the oxidative changes of a colorimetric indicator with a reference standard. In some embodiments, the comparing is conducted by visually inspecting the color of the indicator and one or more colors graphically and/or textually indicated on the reference standard. In further embodiments, the comparing is conducted using spectrophotometric methods such as absorption or emission spectroscopy. In yet further embodiments, the comparing is conducted using electrochemical methods, whereby electrochemical changes in the liquid component of the indicator (when in liquid form) are detected with an electrode.

In some embodiments, color of the indicator can be measured or monitored by automated methods, thereby facilitating assessment of large quantities of articles for oxidative changes. The automated methods can employ spectrophotometric techniques, as described above, for detecting color or electrochemical techniques for detecting electrochemical changes. For example, the automated system could detect absorption or emission at a preselected wavelength, then compare this data with a standard, and use the comparison information to signal whether the article meets a certain threshold of acceptability. In some embodiments, devices such as digital cameras or computer scanners can allow automatated evaluation of products using a computer.

The present invention further provides a liquid pharmaceutical composition which includes a) an active pharmaceutical ingredient (API); b) a calorimetric indicator comprising betalain; and c) a liquid excipient.

The present invention further provides a solid pharmaceutical composition which includes a) an active pharmaceutical ingredient (API); b) a colorimetric indicator comprising betalain; and c) a solid excipient.

The present invention further provides a liquid or solid pharmaceutical composition which includes a) a natural product; b) a calorimetric indicator comprising betalain; and c) and optionally a liquid or solid excipient.

The API in the solid and liquid pharmaceutical compositions of the invention can be any API, such as those which are subject to oxidative degradation, particularly upon exposure to air or ambient environmental conditions. The term “active pharmaceutical ingredient” or “API” is used as known in the art and refers to any substance that is represented for use in a drug and that, when used in the manufacturing, processing, or packaging of a drug, becomes an active ingredient or a finished dosage form of the drug. Such substances are typically intended to furnish pharmacological activity or other direct effect in the diagnosis, cure, mitigation, treatment or prevention of disease, or to affect the structure and function of the body of humans or other animals. APIs include substances manufactured by processes such as (1) chemical synthesis; (2) fermentation; (3) recombinant DNA or other biotechnology methods; (4) isolation/recovery from natural sources; or (5) any combination of these processes. Examples of suitable APIs include small molecule drugs such as ibuprofen, tetracycline, statins, amphoteracin B, hydroquinone, tetrazepam, and the like. Other suitable APIs include proteins such as antibodies or nucleic acids such as antisense molecules or interference RNA. In some embodiments, the API is an FDA approved drug substance. Additionally, natural products can be used in the solid and liquid formulations of the invention described above. Suitable natural products are listed herein.

The solid and liquid pharmaceutical compositions can be in prepared in any form including tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. The compositions can be prepared in dosage forms for oral, parenteral, or other delivery mode. The term “dosage form” refers to a physically discrete unit suitable as a unitary dosage for human subjects and other mammals, each unit containing a predetermined quantity of active material (e.g., API) calculated to produce the desired therapeutic effect, in association with a suitable pharmaceutical excipient. Oral dosage forms include, for example, pill forms such as tablets and capsules. In some embodiments, the solid or liquid pharmaceutical composition is contained within a capsule such as a gel capsule. The gel capsule is preferably transparent or includes a transparent window through which the color of the composition can be viewed or read (such as by eye or by spectroscopic means). Color change in either of the liquid or solid formulations can indicate exposure of the API to oxidative conditions, thereby suggesting potential degradation.

The amount of API provided in the dosage forms of the invention can range from about 0.1 mg to about 5 g, about 1 mg to about 1 g, about 10 mg to about 800 mg, or about 50 mg to about 500 mg. In some embodiments, the dosage form contains about 50, about 100, about 200, about 250, about 300, about 350, about 400, about 450, about 500, or greater than about 500 mg of API such as ibuprofen.

Any pharmaceutically acceptable excipient can be used in the pharmaceutical formulations of the present invention. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Examples of pharmaceutically acceptable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. For liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Betalain can be present in the liquid and solid pharmaceutical compositions of the invention in an amount suitable for detection of its color change upon exposure of the formulation to oxidative conditions that would subject the API, natural product, or other degradable ingredient to potential oxidative degradation. As betalain is strongly colored, only a relatively small amount may be needed. In some instances, where betalain is used as a sacrifical reductant in addition to its indicator ability, more may be used. Accordingly, the solid and liquid formulations of the invention can include less than about 10, less than about 5, less than about 2, less than about 1, less than about 0.8, less than about 0.5, less than about 0.3, less than about 0.2, or less than about 0.1% by weight of betalain. In some embodiments, solid and liquid formulations of the invention contain 1 to about 10%, 1 to about 5% or 2 to about 5% betalain by weight.

Betalain and other pigments can be quantified by a number of different techniques. To find the molar absorptivity or molar fluorescence of a pigment, it can be first purified, and then subjected to either a chemical titration or mass spectroscopy to determine the number of molecules per volume of solution. This solution can then be assessed by the analytical method of choice and a calculation of detector response per mole is calculated. After these steps have been completed, subsequent pigment solutions can be quantified without the use of mass spectroscopy or absolute purity. One example of quantification for betalains is the use of a UV-visible spectrophotometer which monitors the absorbance maxima for a particular betalain such as betanin.

In some embodiments, the liquid and solid compositions of the invention further include a primary reductant. The amount primary reductant in the composition can range from about 0 to about 100, about 0 to about 50, about 0 to about 25, about 0 to abut 15, about 0 to about 10, about 0 to about 5, about 0 to about 3, about 0 to about 2, or about 0 to about 1, % by weight of the total composition depending upon the longevity desired and the limitations of product formulation. In some embodiments, some primary reductants such as ascorbic acid have established quantities for dietary intake and can be formulated to provide 100% or less of the RDA (recommended daily allowance). Because larger doses of ascorbic acid are generally harmless to the consumer, more than 100% RDA can be included if a very long shelf-life is desired, or if the formulation will be in conditions that favor rapid oxidation.

In some embodiments, the liquid and solid compositions of the invention further include an electrolyte. Typically, the liquid and solid compositions of the invention may already have electrolytic properties; however, additional electrolyte can be added to modulate the electrolytic properties of the composition. Electrolytic properties can be measured by resistivity which is the resistance to electricity flow. If the indicator is in direct contact with the article, low resistivity will typically favor accurate determination of preservative remaining in the article. In some embodiments, a pharmaceutical and indicator pigment will be placed together in solution with ions (e.g., electrolyte) thereby providing an efficient conducting media which rapidly and accurately communicates oxidative changes to all components. In systems with higher resistivity, the indicator can be use in the capacity of a calibrated indicator where, for example, the article contains an amount primary reductant sufficient for simultaneous oxidative changes in the article and indicator. In some embodiments, compositions of the invention contain 0.1 to about 10, about 1 to about 5, about 1 to about 3 or about 1 to about 2% electrolyte by weight.

The liquid and solid pharmaceutical compositions of the invention can be prepared by routine formulation, compounding or other methods.

The present invention further provides packaged foods which include food enclosed within a packaging and a calorimetric indicator containing betalain which is also enclosed within the packaging together with the food. In some embodiments, the indicator is separate from the food, meaning that the indicator does not mix with the food and its consumption is not necessary. As betalain is water soluble, the indicator can be separated from the food by a barrier such as a permeable membrane which permits the passage of oxygen and other oxidizing species, but prohibits the passage of betalain. For example the indicator can be enclosed within a membrane pouch or container which is optionally transparent or has a transparent window to facilitate detection of color change. In some embodiments, the indicator is in solid form which is separated from the food by a permeable barrier as described above. In further embodiments, the solid form includes an indicator which is affixed to a solid support.

In some embodiments, the packaged food has an intended shelf life without refrigeration of greater than about 1 year, greater than about 2 years, greater than about 3 years, or greater than about 4 years. In further embodiments, the food is hermetically sealed within its packaging. In yet further embodiments, the packaged food includes a reference standard whereby comparison of the color of the indicator with the reference standard is determinative of the oxidative changes in the food. An example packaged food is a meal-ready to-eat (MRE).

The (MRE) is typically designed to sustain an individual engaged in heavy activity such as military training or during actual military operations when normal food service facilities are not available. The MRE is a self-contained operational ration consisting of a full meal packed in, for example, a flexible meal bag (e.g., retort packaging) or tray container. The full bag or container is preferably lightweight and typically fits-easily into military field clothing pockets. In some embodiments, each MRE contains an entrée such a meat and a variety of other components selected to complement each entrée as well as provide necessary nutrition. Example components include white rice, fruits, bakery items, crackers, spreads, beverages, snacks, candy, hot sauce, noodles, and the like. The fruits can be applesauce, pears, peaches, pineapple, or strawberries. The bakery items include fudge brownie, cookies, fruit bars, toaster pastries, and cake. Each meal can also contain an accessory packet. The contents of one MRE meal bag can provide about 800 to about 2500, about 1000 to about 1500, or about 1250 kilocalories.

The present invention further provides methods of purifying betalain by employing cross-flow membrane filtration technology. Cross-flow membrane filtration technology employs a type of filter module or filter cassette that comprises a porous filter element across a surface of which the liquid medium to be filtered is flowed in a tangential flow fashion, for permeation through the filter element of selected component(s) of the liquid medium. The shear force exerted on the filter element (separation membrane surface) by the flow of the liquid medium serves to oppose accumulation of solids on the surface of the filter element. Useful cross-flow filters include microfiltration, ultrafiltration, nanofiltration and reverse osmosis filter systems. The cross-flow filter can comprise a multiplicity of filter sheets (filtration membranes) in an operative stacked arrangement, e.g., wherein filter sheets alternate with permeate and retentate sheets, and as a liquid to be filtered flows across the filter sheets, impermeate (non-permeating) species, e.g., solids or high-molecular-weight species of diameter larger than the filter sheet's pore size(s), are retained and enter the retentate flow, and the liquid along with any permeate species diffuse through the filter sheet and enter the permeate flow. For example, a plant extract containing betalain can undergo one or more microfiltration, ultrafiltration, nanofiltration, reverse osmosis, demineralization, or combination thereof using cross-flow filtration. Typically, the plant extract is successively filtered first through coarse and then finer and finer membranes, thereby separating the components of the extract largely by size. The fraction containing betalain can be identified and/or quantified by visual or spectroscopic methods (such as absorption, emission, or fluorescence).

The filtration process allows ultrafiltration, nanofiltration, and reverse osmosis membranes to purify components of plant extract by size while concentrating the products. After a semi-pure, concentrated betalain is produced it can be quantified and standardized. Methods for quantifying betalain are well known in the art and include absorbance measurements by spectrophotometer, mass spectroscopy, HPLC, electrical degradation by electrode, and other techniques. This methodology allows the utilization of plentiful plant extracts having relatively low concentrations of betalain while allowing separation and sale of other valuable products made by the plant.

Cross-flow filtration technology is commercially available and described, for example in, U.S. Pat. Nos. 4,867,876; 4,882,050; 5,034,124; 5,049,268; 5,232,589; 5,342,517; 5,593,580; 5,868,930; the disclosures of each of which are incorporated herein by reference in their entireties. An application of cross-flow technology to separation of components of milk is described in U.S. Pat. No. 6,852,352, the disclosure of which is incorporated herein by reference in its entirety.

The purification process can further include one or more fermentation steps which can, for example, selectively remove carbohydrates and other unwanted organic and inorganic components from the extract. Fermentation can be conducted prior to filtration, after filtration, or at an intermediate stage in the filtration process. Fermentation techniques are routine in the art. An example fermentation process for purifying betalain is described in, for example, U.S. Pat. No. 4,027,042, which is incorporated herein by reference in its entirety.

In order that the invention disclosed herein may be more efficiently understood, examples are provided below. It should be understood that these examples are for illustrative purposes only and are not to be construed as limiting the invention in any manner.

### EXAMPLES

### Example 1

### Liquid-Filled Capsules and Packaging

This example illustrates a liquid-filled gelatin capsule containing a low resistivity excipient, betalain, ascorbic acid, and a pharmaceutical or natural product (e.g., API, nutraceutical, vitamin, or herbal remedy). The capsule is sealed after filling to prevent the free flow of oxygen into the capsule. The capsule is packaged in a container made of a material that is substantially impermeable to oxygen (e.g., glass). Desiccants and oxygen scavengers are optionally included within the packaging. The packaging is sealed under inert gas. The container is substantially transparent and made of glass, polycarbonate, or plastic. This container is covered with an opaque label, leaving the bottom and/or upper edge of the bottle uncovered. Optionally, any transparent part of the container is covered with a removable/replaceable opaque tab. This tab would impede light into the container until evaluation is required and it can be peeled away allowing the consumer to see inside. Optionally, two transparent areas on opposite sides of the package are provided to allow the admission as well as egress of light. On the label or the tab is placed a calorimetric indicator reference bar, which serves as the color reference standard allowing the consumer a direct side-by-side comparison of the printed color bar to the indicator's color. This calorimetric bar will also have printed instructions that allow the consumer to understand the functionality of the indicator. This could also be augmented by pictograms that would allow illiterate or foreign language speakers to grasp the concept that the color on the label corresponds to the state of the product and its suitability for consumption.

### Example 2

### Pain-Relieving Liquid Pharmaceutical Composition

A liquid pharmaceutical composition of the invention contains 2-5% by weight betalain and 100 mg ibuprofen in 5 mL of liquid matrix containing acesulfame potassium, citric acid, corn starch, flavors, glycerin, natural and artificial flavors, polysorbate 80, purified water, sodium benzoate, sucrose, and xanthan gum.

### Example 3

### Pain-Relieving Solid Pharmaceutical Composition

A solid pharmaceutical composition of the invention contains 2-5% by weight betalain; 200 mg ibuprofen in solid excipient containing carnauba wax, corn starch, hypromellose, iron oxide, polydextrose, polyethylene glycol, silicon dioxide, stearic acid and titanium dioxide. The solid composition can be in the form of a tablet.

### Example 4

### Ibuprofen Gelcaps

Each gelcap encloses a liquid formulation having 2-5% by weight betalain, 200 mg ibuprofen, as well as benzyl alcohol, butylparaben, castor oil, cellulose, corn starch, edetate calcium disodium, gelatin, hypromellose, iron oxide, methylparaben, povidone, propylparaben, silicon dioxide, sodium lauryl sulfate, sodium propionate, sodium starch glycolate, and titanium dioxide.

### Example 5

### Betalain Disposed on Solid Support

Betalain is chemically affixed to hydroxypropylmethylcellulose or caroboxymethylcellulose in a 1:1 weight ratio. The resulting betalain on solid support is placed within a sealed food package or incorporated into a topical skin product.

### Example 6

### Formulations for Topical Application

### Cream

One gram of cream contains 1 mg of hydrocortisone butyrate and 2-5% by weight betalain in a hydrophilic base containing cetostearyl alcohol, ceteth-20, mineral oil, white petrolatum, citric acid, sodium citrate, propylbaraben, butylparaben, and purified water.

### Ointment

One gram of ointment contains 1 mg of hydrocortisone butyrate and 2-5% by weight betalain in a base containing mineral oil and polyethylene.

### Example 7

### Calibrated Indicator with a Percentage of Colored Proxies

This example illustrates estimation of product quality using proxies. About 10% of the number of pills in a container contain betalain and serve as proxies to indicate oxidation status of pills which do not contain betalain. The proxy and non-proxy pills both contain ascorbic acid and are characterized using HPLC and mass spectroscopy to confirm substantial equivalence of the rates of oxidation.

### Example 8

### Calibrated Indicator with a Package-Integrated Colored Proxy

This example illustrates estimation of product quality using proxies. A container of pills can include betalain and ascorbic acid infused into wax paper serving as a gasket in the lid of the container. The wax is perforated in the center of the disk without compromising the gasket function to allow more exposure of the betalain to the interior environment of the container. The pills contain ascorbic acid as a primary reductant. The betalain-infused wax and pills are calibrated for substantially equal rates of oxidative degradation. The rates of oxidation and quantity of preservative are confirmed both before full production and during production using HPLC and mass spectroscopy.

### Example 9

### Topically Applied Sunscreen and Sterilizing Agent

This example provides an illustration of betalain indicator in topically applied consumer products. Sunscreen with ethanol as a sterilizing agent are combined with moisturizing emollients, a primary reductant, and betalain. The betalain allows visualization of complete coverage, acts as a revealing agent for adhered particles, and provides cosmetic coverage of freckles, veins, etc. Removal of only the betalain is carried out by immersion in water which dissolves the betalain, leaving clean, sterilized skin with protection from UV radiation. Soap and water are used to remove all product from the skin.

### Example 10

### Antitoxins and Vaccines

This example serves to illustrate the use of indicator in antitoxin and vaccine formulations. An antitoxin or vaccine can be formulated for injection in sterilized aqueous solution containing a pharmaceutically acceptable preservative and 1-5% by weight betalain.

### Example 11

### Further Uses of Betalain as an Indicator

Betalain is used to detect the depletion of the zinc coating of galvanized steel by layering the betalain on the metal surface. Absence or depletion of the protective zinc coating would result in betalain color change, indicating exposure of the steel to oxidation.

Betalain is used to determine the effectiveness of a charcoal filter used to remove dissolved oxidizing species (e.g., chlorine) from a liquid reagent sample. Betalain is added to a filtered sample, and the resulting color, when compared with an unfiltered sample or sample with known quantity of oxidizing species, can indicate the effectiveness of the filter.

Various modifications of the invention, in addition to those described herein, will be apparent to those skilled in the art from the foregoing description. Such modifications are also intended to fall within the scope of the appended claims. Each reference cited in the present application, including patents, published applications, and journal articles, is incorporated herein by reference in its entirety.

What is claimed is:

1. A system for determining the presence or absence of oxidative changes in an article, comprising:
a) an article which is subject to degradation or spoilage in the presence of air or oxidative conditions;
b) a calorimetric indicator comprising a colored compound which changes color upon oxidation; wherein said indicator is exposed to substantially the same environmental conditions as said article; and
c) a reference standard, whereby comparison of the color of said indicator with said reference standard determines the presence or absence of oxidative changes in said article.

2. The system of claim 1 wherein said article and said indicator are combined together.

3. The system of claim 2 wherein said article and said indicator are enclosed together within a packaging.

4. The system of claim 3 wherein said reference standard is disposed on said packaging or referred to on the labeling of said packaging.

5. The system of claim 1 wherein said indicator is derived from a natural source.

6. The system of claim 1 wherein said indicator comprises a betalain.

7. The system of claim 1 wherein said indicator comprises a betacyanin or derivative thereof.

8. The system of claim 1 wherein said indicator comprises a betaxanthin or derivative thereof.

9. The system of claim 1 wherein said indicator is derived from fungi of the family Amanita or plants of the order Caryophyllales.

10. The system of claim 1 wherein said indicator is derived from a plant of the family Chenopodiaceae, Cactaceae, Amaranthaceae, Nyctaginaceae, Aizoaceae, Basellaceae, Didieraceae, Phytolaccaceae, or Portulaceae.

11. The system of claim 1 wherein said indicator is derived from amaranth, Malabar spinach fruit, red beets, prickly pear, Phytolacca americana, Capitatum berries (Chenopodium capitatum), or hottentot fig (Carpobrotus edulis).

12. The system of claim 1 wherein said indicator is in liquid form.

13. The system of claim 1 wherein said indicator is in solid form.

14. The system of claim 1 wherein said indicator is disposed on a solid substrate.

15. The system of claim 1 further comprising an electrolyte.

16. The system of claim 1 further comprising an electrolyte which is in contact with both said indicator and said article.

17. The system of claim 1 further comprising a primary reductant.

18. The system of claim 17 wherein said primary reductant is a stronger reductant than said colored compound or a substantially equal reductant to said colored compound.

19. The system of claim 17 wherein said primary reductant is ascorbic acid, catenin, tocopherol, tocotrienol, or mixture thereof.

20. The system of claim 1 wherein said article is a pharmaceutical, a natural product, a food, or a skin treatment.

21. The system of claim 1 wherein said article is a pharmaceutical.

22. The system of claim 21 wherein said pharmaceutical comprises ibuprofen, tetracycline, a statin, amphoteracin B, hydroquinone, tetrazepam, or mixture thereof.

23. The system of claim 1 wherein said reference standard comprises a graphic indication, textual indication, or combination thereof of the presence or absence of oxidative changes in said article.

24. The system of claim 23 wherein said graphic indication comprises one or more colors corresponding to substantially unoxidized, substantially oxidized, or partially oxidized indicator.

25. The system of claim 24 wherein said textual indication comprises one or more words or phrases describing the color of substantially unoxidized, substantially oxidized, or partially oxidized indicator.

26. The system of claim 1 wherein said reference standard comprises spectrophotometric information.

27. A system for determining the presence or absence of oxidative changes in a pharmaceutical, comprising:
a) a pharmaceutical composition containing a component which is subject to degradation in the presence of air or oxidative conditions;
b) a calorimetric indicator comprising a betalain, wherein said indicator is exposed to substantially the same environmental conditions as said pharmaceutical composition; and
c) optionally a reference standard, whereby comparison of the color of said indicator with said reference standard determines the presence or absence of oxidative changes in said pharmaceutical.

28. The system of claim 27 wherein said component is an active pharmaceutical ingredient (API).

29. A liquid pharmaceutical composition comprising:
a) an active pharmaceutical ingredient (API);
b) a calorimetric indicator comprising a betalain; and
c) a liquid excipient.

30. The liquid pharmaceutical composition of claim 29 which is in oral dosage form.

31. The liquid pharmaceutical composition of claim 30 wherein said oral dosage form is a capsule containing said liquid pharmaceutical.

32. The liquid pharmaceutical composition of claim 31 wherein said capsule is transparent.

33. The liquid pharmaceutical composition of claim 29 further comprising an electrolyte.

34. The liquid pharmaceutical composition of claim 29 further comprising a primary reductant.

35. The liquid pharmaceutical composition of claim 29 wherein said API is ibuprofen.

36. A solid pharmaceutical composition comprising:
a) an active pharmaceutical ingredient (API);
b) a calorimetric indicator comprising a betalain; and
c) a solid excipient.

37. The solid pharmaceutical composition of claim 36 which is in oral dosage form.

38. The solid pharmaceutical composition of claim 37 which is contained within a capsule.

39. The solid pharmaceutical composition of claim 38 wherein said capsule is transparent.

40. The solid pharmaceutical composition of claim 36 further comprising an electrolyte.

41. The solid pharmaceutical composition of claim 36 further comprising a primary reductant.

42. A packaged food comprising:
a) food enclosed within a packaging; and
b) a calorimetric indicator which is enclosed within said packaging together with said food, wherein said indicator is separate from said food, and wherein said indicator comprises a betalain.

43. The packaged food of claim 42 wherein said packaged food has an intended shelf life without refrigeration of greater than about 1 year.

44. The packaged food of claim 42 wherein said food is hermetically sealed within said packaging.

45. The packaged food of claim 42 wherein said indicator is in solid form.

46. The packaged food of claim 45 wherein said indicator is disposed on a solid substrate.

47. The packaged food of claim 42 further comprising a reference standard whereby comparison of the color of said indicator with said reference standard is determinative of the presence or absence of oxidative changes in said food.

48. The packaged food of claim 42 which is a meal-ready to-eat (MRE).

49. A method of determining the presence or absence of oxidative changes in an article, comprising comparing the oxidative changes of a calorimetric indicator comprising a betalain with a reference standard, wherein said indicator is exposed to substantially the same environmental conditions as said article; whereby said comparison indicates the presence or absence of oxidative changes in of said, article.

50. The method of claim 49 wherein said comparing is conducted by visually inspecting the color of said indicator and visually inspecting one or more colors graphically or textually indicated on said reference standard.

51. The method of claim 49 wherein said comparing is conducted using spectrophotometric methods.

52. The method of claim 49 wherein said comparing is conducted using electrochemical methods.

53. The method of claim 49 wherein said comparing is automated.

54. A method of purifying betalain comprising filtering a plant extract comprising betalain, wherein said filtering comprises cross-flow membrane filtration technology.

55. The method of claim 54 wherein said plant extract undergoes microfiltration, ultrafiltration, nanofiltration, reverse osmosis, demineralization, or combinations thereof.

56. The method of claim 54 further comprising subjecting said extract or partially purified form thereof to fermentation.

57. The method of claim 54 further comprising quantifying said betalain using spectroscopy.
